(19) United States
(12) Patent Application Publication
(10) Pub. No.: US 2026/0256377 A1
(43) Pub. Date: Sep. 3, 2026

(54) ALCOHOL DETECTION APPARATUS

(71) Applicant: SUBARU CORPORATION, Tokyo (JP)

(72) Inventors: Satoru IWATA, Tokyo (JP); Hideaki ASANO, Tokyo (JP); Shinichi MIYAMOTO, Tokyo (JP); Hideyuki KARASAWA, Tokyo (JP)

(21) Appl. No.: 18/881,789

(22) PCT Filed: Apr. 25, 2023
(86) PCT No.: PCT/JP2023/016253

§ 371 Date: Jan. 7, 2025

(51) Int. Cl. A61B 5/08; A61B 5/00; A61B 5/097; B60R 16/023

(52) U.S. Cl. CPC A61B 5/082; A61B 5/097; A61B 5/4845; A61B 5/6893; B60R 16/0231; A61B 2503/22

(57) ABSTRACT

An alcohol detection apparatus according to an aspect of the disclosure includes an alcohol sensor and a control circuit. The alcohol sensor is configured to detect an alcohol component included in exhaled breath of an occupant of a vehicle. The control circuit is configured to, based on a detection result of a door sensor configured to detect an open or closed state of each of doors of the vehicle, control the alcohol sensor not to perform an initialization operation in a period in which one or more of the doors are in an open state, and control the alcohol sensor to perform the initialization operation after all the doors enter the closed state.

### TECHNICAL FIELD

The disclosure relates to an alcohol detection apparatus configured to detect an alcohol component included in exhaled breath of an occupant.

### BACKGROUND ART

Some vehicles have an apparatus that determines whether an occupant has drunk alcohol. For example, Patent Literature 1 discloses an apparatus that prompts detection of alcohol with a door at a driver's seat closed when the door at the driver's seat is open.

### CITATION LIST

### Patent Literature

Patent Literature 1: Japanese Unexamined Patent Application Publication No. 2009-258872

### SUMMARY OF INVENTION

An aspect of the disclosure provides an alcohol detection apparatus including an alcohol sensor and a control circuit. The alcohol sensor is configured to detect an alcohol component included in exhaled breath of an occupant of a vehicle. The control circuit is configured to, based on a detection result of a door sensor configured to detect an open or closed state of each of doors of the vehicle, control the alcohol sensor not to perform an initialization operation in a period in which one or more of the doors are in an open state, and control the alcohol sensor to perform the initialization operation after all the doors enter the closed state.

An aspect of the disclosure provides an alcohol detection apparatus including an alcohol sensor and a control circuit. The alcohol sensor is configured to detect an alcohol component included in exhaled breath of an occupant of a vehicle. The control circuit is configured to, based on a detection result of a door lock sensor configured to detect a lock state of each of doors of the vehicle, control the alcohol sensor not to perform an initialization operation in a period in which the lock state of each of one or more of the doors is an unlocked state, and control the alcohol sensor to perform the initialization operation after the lock state of each of all the doors becomes the locked state.

### BRIEF DESCRIPTION OF DRAWINGS

The accompanying drawings are included to provide a further understanding of the disclosure, and are incorporated in and constitute a part of this specification. The drawings illustrate embodiments and, together with the specification, serve to explain the principles of the disclosure.

FIG. 1 is an explanatory diagram illustrating a configuration example of a vehicle according to one embodiment of the disclosure.

FIG. 2 is a block diagram illustrating a configuration example of the vehicle illustrated in FIG. 1.

FIG. 3 is an explanatory diagram illustrating a configuration example of a vehicle compartment of the vehicle illustrated in FIG. 1.

FIG. 4 is an explanatory diagram illustrating a configuration example of an alcohol sensor illustrated in FIG. 2.

FIG. 5 is a flowchart illustrating an operation example of an alcohol detection apparatus illustrated in FIG. 2.

FIG. 6 is a block diagram illustrating a configuration example of a vehicle according to a modification example.

FIG. 7 is a flowchart illustrating an operation example of the alcohol detection apparatus illustrated in FIG. 2.

FIG. 8A is an explanatory diagram illustrating an operation state of an alcohol sensor according to another modification example.

FIG. 8B is an explanatory diagram illustrating another operation state of the alcohol sensor according to the other modification example.

FIG. 9 is a flowchart illustrating an operation example of an alcohol detection apparatus according to another modification example.

### MODES FOR CARRYING OUT THE INVENTION

It is desired that an alcohol detection apparatus achieve a high detection accuracy of an alcohol component, and a further improvement in the detection accuracy is expected.

It is desirable to provide an alcohol detection apparatus that makes it possible to increase a detection accuracy of an alcohol component.

In the following, some example embodiments of the disclosure are described in detail with reference to the accompanying drawings. Note that the following description is directed to illustrative examples of the disclosure and not to be construed as limiting to the disclosure. Factors including, without limitation, numerical values, shapes, materials, components, positions of the components, and how the components are coupled to each other are illustrative only and not to be construed as limiting to the disclosure. Further, elements in the following example embodiments which are not recited in a most-generic independent claim of the disclosure are optional and may be provided on an as-needed basis. The drawings are schematic and are not intended to be drawn to scale. Throughout the present specification and the drawings, elements having substantially the same function and configuration are denoted with the same reference numerals to avoid any redundant description. In addition, elements that are not directly related to any embodiment of the disclosure are unillustrated in the drawings.

### Embodiment

### Configuration Example

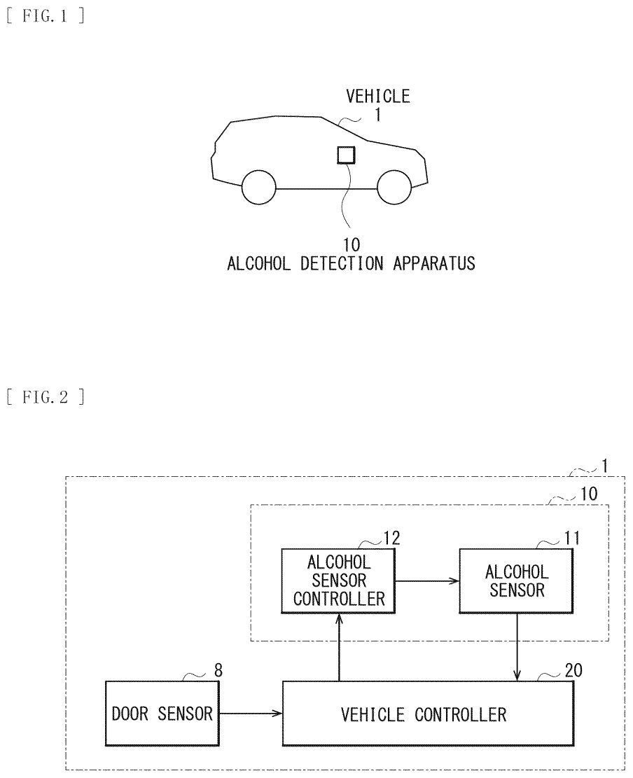

FIGS. 1 and 2 illustrate a configuration example of a vehicle 1 provided with an alcohol detection apparatus according to an embodiment. The vehicle 1 includes an alcohol detection apparatus 10, a door sensor 8, and a vehicle controller 20.

The alcohol detection apparatus 10 includes an alcohol sensor 11 and an alcohol sensor controller 12.

The alcohol sensor 11 is configured to detect an alcohol component included in exhaled breath of a driver. The alcohol sensor 11 detects the alcohol component included in the exhaled breath of the driver, based on, for example, blowing of a breath of the driver toward a later-described inlet 11A of the alcohol sensor 11. The alcohol sensor 11 becomes available by performing an initialization operation of adjusting what is called a zero point. That is, the alcohol sensor 11 is calibrated by the initialization operation to become able to detect the alcohol component at a high detection accuracy. The initialization operation of the alcohol sensor 11 is controlled by the alcohol sensor controller 12.

The alcohol sensor controller 12 is configured to control the operation of the alcohol sensor 11 based on an instruction from the vehicle controller 20. Specifically, the alcohol sensor controller 12 controls the operation of the alcohol sensor 11 based on an open or closed state of each of doors of the vehicle 1, as will be described later.

The door sensor 8 is configured to detect the open or closed state of each of the doors of the vehicle 1. Examples of the doors include a door at a driver's seat, a door at a passenger seat, left and right doors at rear seats, and a rear gate. The door sensor 8 supplies a detection result regarding the open or closed state of each of the doors to the vehicle controller 20.

The vehicle controller 20 includes, for example, an electronic control unit (ECU), and is configured to control the vehicle 1. In this example, the vehicle controller 20 supplies information regarding the open or closed state of each of the doors to the alcohol sensor controller 12, based on the detection result of the door sensor 8. Based on a detection result of the alcohol sensor 11, the vehicle controller 20 allows the vehicle 1 to travel when an amount of the alcohol component included in the exhaled breath of the driver is equal to or less than a predetermined amount. The vehicle controller 20 prevents the vehicle 1 from traveling when the amount of the alcohol component included in the exhaled breath of the driver is greater than the predetermined amount. Specifically, the vehicle controller 20 does not allow a power source such as an engine or a motor to start, for example, when the amount of the alcohol component included in the exhaled breath of the driver is greater than the predetermined amount.

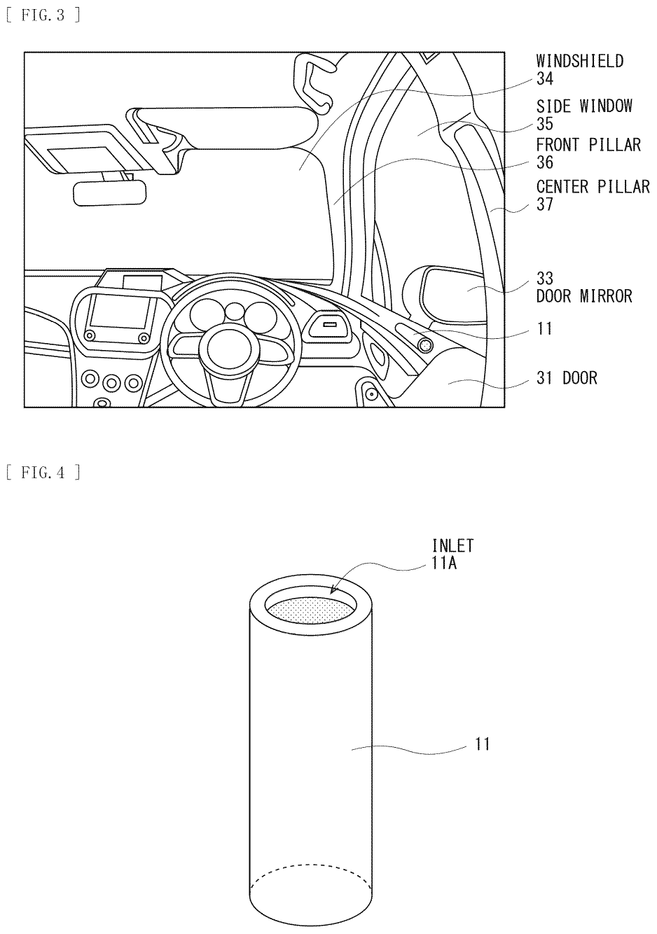

FIG. 3 illustrates a configuration example of a vehicle compartment of the vehicle 1. The vehicle 1 has the driver's seat on the right side of the vehicle 1. Note that the disclosure is not limited thereto, and the driver's seat may be provided on the left side of the vehicle 1. The vehicle 1 includes a door 31, a door mirror 33, a windshield 34, a side window 35, a front pillar 36, and a center pillar 37. The door 31 is provided on a side surface of the vehicle 1, and allows the driver to get on and off the vehicle 1. In this example, the alcohol sensor 11 is mounted on an inner panel of the door 31 opposed to the vehicle compartment.

FIG. 4 illustrates a configuration example of the alcohol sensor 11. The alcohol sensor 11 includes the inlet 11A. The driver seated on the driver's seat blows his/her breath toward the inlet 11A, and the alcohol sensor 11 thereby detects the alcohol component included in the exhaled breath of the driver.

The door mirror 33 is provided on an outer surface of the door 31. The windshield 34 is provided at the front of the vehicle compartment of the vehicle 1. The side window 35 is provided in the door 31. The front pillar 36 supports a roof of the vehicle 1 at an end of the windshield 34. The center pillar 37 supports the roof of the vehicle 1 in the vicinity of the middle of the side surface of the vehicle 1.

In the alcohol detection apparatus 10, the alcohol sensor controller 12 controls the operation of the alcohol sensor 11 to prevent the alcohol sensor 11 from performing the initialization operation, for example, in a period in which one or more of the doors of the vehicle 1 are in an open state. The alcohol sensor controller 12 controls the operation of the alcohol sensor 11 to cause the alcohol sensor 11 to perform the initialization operation, for example, after all the doors of the vehicle 1 enter a closed state. This allows the alcohol detection apparatus 10 to perform the initialization operation while the door is closed, without being affected by, for example, impact, wind pressure, or an atmospheric pressure change in the vehicle caused by opening and closing of the door. Consequently, it is possible for the alcohol detection apparatus 10 to achieve an increased detection accuracy.

Here, the alcohol sensor 11 corresponds to a specific example of an “alcohol sensor” in one embodiment of the disclosure. The alcohol sensor controller 12 corresponds to a specific example of a “control circuit” in one embodiment of the disclosure. The vehicle 1 corresponds to a specific example of a “vehicle” in one embodiment of the disclosure.

### Operation and Workings

Next, an operation and workings of the alcohol detection apparatus 10 according to the embodiment will be described.

### Overview of Overall Operation

The operation of the vehicle 1 will be described first with reference to FIG. 2. The alcohol sensor 11 detects the alcohol component included in the exhaled breath of the driver. The alcohol sensor controller 12 controls the operation of the alcohol sensor 11 based on an instruction from the vehicle controller 20. The door sensor 8 detects the open or closed state of each of the doors of the vehicle 1. The vehicle controller 20 supplies the information regarding the open or closed state of each of the doors to the alcohol sensor controller 12, based on the detection result of the door sensor 8. Based on the detection result of the alcohol sensor 11, the vehicle controller 20 allows the vehicle 1 to travel when the amount of the alcohol component included in the exhaled breath of the driver is equal to or less than the predetermined amount. The vehicle controller 20 prevents the vehicle 1 from traveling when the amount of the alcohol component included in the exhaled breath of the driver is greater than the predetermined amount.

### Detailed Operation

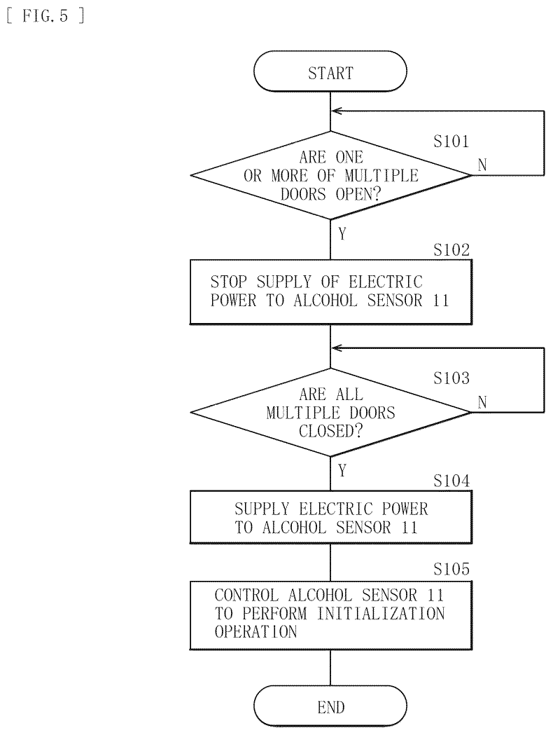

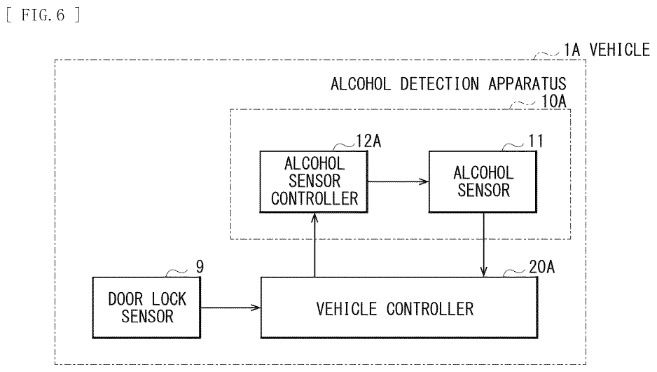

FIG. 5 illustrates an operation example of the alcohol detection apparatus 10. The alcohol detection apparatus 10 is supplied with the information regarding the open or closed state of each of the doors of the vehicle 1 from the vehicle controller 20. The alcohol detection apparatus 10 controls the operation of the alcohol sensor 11 based on the information. The following describes this operation in detail.

First, the alcohol sensor controller 12 determines whether one or more of the doors are open, based on the information regarding the open or closed state of each of the doors supplied from the vehicle controller 20 (step S101). If all the doors are closed (“N” in step S101), the alcohol sensor controller 12 repeats the process of step S101 until one or more of the doors are opened.

If one or more of the doors are open in step S101 (“Y” in step S101), the alcohol sensor controller 12 stops supply of electric power to the alcohol sensor 11 (step S102). In this manner, an operation mode of the alcohol detection apparatus 10 becomes a standby mode of waiting for detection of the alcohol component.

Next, the alcohol sensor controller 12 checks whether all the doors are closed, based on the information regarding the open or closed state of each of the doors supplied from the vehicle controller 20 (step S103). If one or more of the doors are still open (“N” in step S103), the alcohol sensor controller 12 repeats the process of step S103 until all the doors are closed.

If all the doors are closed in step S103 (“Y” in step S103), the alcohol sensor controller 12 supplies electric power to the alcohol sensor 11 (step S104).

Thereafter, the alcohol sensor controller 12 controls the operation of the alcohol sensor 11 to cause the alcohol sensor 11 to perform the initialization operation (step S105). The alcohol sensor 11 is calibrated by the initialization operation to become able to detect the alcohol component at a high detection accuracy. In this manner, the operation mode of the alcohol detection apparatus 10 becomes a detection mode in which the alcohol component is detectable.

This is the end of the process. Thereafter, the driver seated in the driver's seat blows his/her breath toward the inlet 11A of the alcohol sensor 11. The alcohol detection apparatus 10 thus detects the alcohol component included in the exhaled breath of the driver. Based on the detection result of the alcohol sensor 11, the vehicle controller 20 allows the vehicle 1 to travel when the amount of the alcohol component included in the exhaled breath of the driver is equal to or less than the predetermined amount, and prevents the vehicle 1 from traveling when the amount of the alcohol component included in the exhaled breath of the driver is greater than the predetermined amount.

For example, the alcohol sensor controller 12 may supply electric power to the alcohol sensor 11 in a period until the vehicle 1 starts traveling, or may continuously supply electric power to the alcohol sensor 11 in a period in which the vehicle 1 is traveling. For example, in a case of continuously supplying electric power in the period in which the vehicle 1 is traveling, the vehicle controller 20 stops the power source such as the engine or the motor after giving a predetermined warning to the driver, for example, when the amount of the alcohol component included in the exhaled breath of the driver becomes equal to or greater than the predetermined amount while the vehicle 1 is traveling. This allows the alcohol detection apparatus 10 to stop the traveling of the vehicle 1, for example, even if the driver drinks alcohol after starting driving.

As described above, the alcohol detection apparatus 10 includes the alcohol sensor 11 and the alcohol sensor controller 12. The alcohol sensor 11 is configured to detect the alcohol component included in the exhaled breath of an occupant of the vehicle 1. The alcohol sensor controller 12 is configured to, based on the detection result of the door sensor 8 configured to detect the open or closed state of each of the doors of the vehicle 1, control the alcohol sensor 11 not to perform the initialization operation in the period in which one or more of the doors are in the open state, and control the alcohol sensor 11 to perform the initialization operation after all the doors enter the closed state. This helps to increase the detection accuracy of the alcohol component in the alcohol detection apparatus 10.

That is, for example, when the alcohol sensor 11 performs the initialization operation while the door is open, the initialization operation can be performed incorrectly depending on an environmental condition such as wind. In addition, for example, when the alcohol sensor 11 performs the initialization operation at a timing of opening and closing the door, the initialization operation can be performed incorrectly due to, for example, impact, wind pressure, or an atmospheric pressure change in the vehicle caused by opening and closing of the door. If the initialization operation is thus performed incorrectly, the alcohol sensor 11 can be poorly calibrated, which can cause a decrease in the detection accuracy of the alcohol detection apparatus 10.

In contrast, in the alcohol detection apparatus 10 according to the embodiment, in the period in which one or more of the doors are in the open state, supply of electric power to the alcohol sensor 11 is stopped to prevent the alcohol sensor 11 from performing the initialization operation. After all the doors enter the closed state, electric power is supplied to the alcohol sensor 11 to cause the alcohol sensor 11 to perform the initialization operation. Thus, in the alcohol detection apparatus 10, the alcohol sensor 11 performs the initialization operation, for example, after all the doors enter the closed state. This makes it possible to perform the initialization operation at a timing when an environment is stabilized after, for example, impact, wind pressure, or an atmospheric pressure change in the vehicle is caused by opening and closing of the door. Thus, the alcohol sensor 11 is appropriately calibrated in the alcohol detection apparatus 10, which helps to increase the detection accuracy of the alcohol component.

In addition, in the alcohol detection apparatus 10, the alcohol sensor controller 12 is configured to stop supply of electric power to the alcohol sensor 11 in the period in which one or more of the doors are in the open state, and supply electric power to the alcohol sensor 11 in a period in which all the doors are in the closed state. Thus, in the alcohol detection apparatus 10, it is possible to prevent the alcohol sensor 11 from operating, for example, in the period in which one or more of the doors are in the open state, which makes it possible to reduce the possibility of the detection accuracy decreasing due to the environment such as wind. After all the doors enter the closed state, electric power is supplied to the alcohol sensor 11 and the alcohol sensor 11 performs the initialization operation, which makes it possible to perform the initialization operation, for example, immediately before the driver blows his/her breath toward the inlet 11A. Consequently, it is possible to perform the initialization operation and the operation of detecting the alcohol component in a short period in which the environmental condition such as temperature is kept constant, for example, after all the doors enter the closed state. This helps to increase the detection accuracy of the alcohol component.

### Effects

As described above, in the embodiment, the alcohol sensor and the alcohol sensor controller are provided. The alcohol sensor is configured to detect the alcohol component included in the exhaled breath of the occupant of the vehicle. The alcohol sensor controller is configured to, based on the detection result of the door sensor configured to detect the open or closed state of each of the doors of the vehicle, control the alcohol sensor not to perform the initialization operation in the period in which one or more of the doors are in the open state, and control the alcohol sensor 11 to perform the initialization operation after all the doors enter the closed state. This helps to increase the detection accuracy of the alcohol component.

In the embodiment, the alcohol sensor controller is configured to stop supply of electric power to the alcohol sensor in the period in which one or more of the doors are in the open state, and supply electric power to the alcohol sensor in the period in which all the doors are in the closed state. This helps to increase the detection accuracy of the alcohol component.

### Modification Example 1

In the embodiment described above, the operation of the alcohol sensor 11 is controlled based on the open or closed state of the door, but the disclosure is not limited thereto. Alternatively, for example, the operation of the alcohol sensor 11 may be controlled based on a lock state of the door. The following describes a vehicle 1A according to the modification example in detail.

FIG. 6 illustrates a configuration example of the vehicle 1A. The vehicle 1A includes an alcohol detection apparatus 10A, a door lock sensor 9, and a vehicle controller 20A.

The alcohol detection apparatus 10A includes an alcohol sensor controller 12A. The alcohol sensor controller 12A is configured to control the operation of the alcohol sensor 11 based on an instruction from the vehicle controller 20A. The alcohol sensor controller 12A controls the operation of the alcohol sensor 11 based on the lock state of each of the doors of the vehicle 1.

The door lock sensor 9 is configured to detect the lock state of each of the doors of the vehicle 1. The door lock sensor 9 supplies a detection result regarding the lock state of each of the doors to the vehicle controller 20A.

The vehicle controller 20A is configured to control the vehicle 1A. In this example, the vehicle controller 20A supplies information regarding the lock state of each of the doors to the alcohol sensor controller 12A, based on the detection result of the door lock sensor 9. Based on the detection result of the alcohol sensor 11, the vehicle controller 20A allows the vehicle 1 to travel when the amount of the alcohol component included in the exhaled breath of the driver is equal to or less than the predetermined amount, and prevents the vehicle 1 from traveling when the amount of the alcohol component included in the exhaled breath of the driver is greater than the predetermined amount.

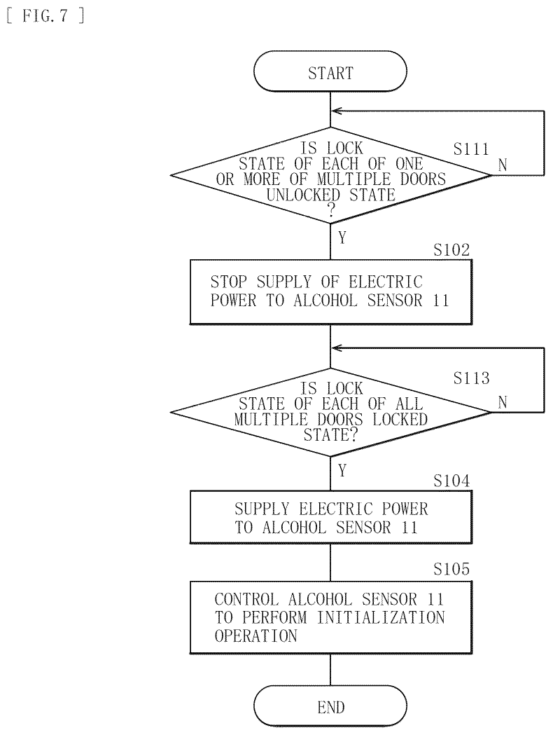

FIG. 7 illustrates an operation example of the alcohol detection apparatus 10A.

First, the alcohol sensor controller 12A checks whether the lock state of each of one or more of the doors is an unlocked state, based on the information regarding the lock state of each of the doors supplied from the vehicle controller 20A (step S111). If the lock state of each of all the doors is a locked state (“N” in step S111), the alcohol sensor controller 12A repeats the process of step S111 until one or more of the doors enter the unlocked state.

If the lock state of each of one or more of the doors is the unlocked state in step S111 (“Y” in step S111), the alcohol sensor controller 12A stops supply of electric power to the alcohol sensor 11 (step S102). In this manner, the operation mode of the alcohol detection apparatus 10A becomes the standby mode of waiting for detection of the alcohol component.

Next, the alcohol sensor controller 12A checks whether the lock state of each of all the doors are the locked state, based on the information regarding the lock state of each of the doors supplied from the vehicle controller 20A (step S113). If the lock state of each of one or more of the doors is still the unlocked state (“N” in step S113), the alcohol sensor controller 12A repeats the process of step S113 until the lock state of each of all the doors becomes the locked state.

If the lock state of each of all the doors is the locked state in step S113 (“Y” in step S113), the alcohol sensor controller 12A supplies electric power to the alcohol sensor 11 (step S104).

Thereafter, the alcohol sensor controller 12A controls the operation of the alcohol sensor 11 to cause the alcohol sensor 11 to perform the initialization operation (step S105). The alcohol sensor 11 is calibrated by the initialization operation to become able to detect the alcohol component at a high detection accuracy. In this manner, the operation mode of the alcohol detection apparatus 10 becomes the detection mode in which the alcohol component is detectable.

This is the end of the process.

The alcohol detection apparatus 10A according to the modification example includes the alcohol sensor 11 and the alcohol sensor controller 12A. The alcohol sensor 11 is configured to detect the alcohol component included in the exhaled breath of the occupant of the vehicle 1A. The alcohol sensor controller 12A is configured to, based on the detection result of the door lock sensor 9 configured to detect the lock state of each of the doors of the vehicle 1A, control the alcohol sensor 11 not to perform the initialization operation in a period in which the lock state of each of one or more of the doors is the unlocked state, and control the alcohol sensor to perform the initialization operation after the lock state of each of all the doors becomes the locked state. Thus, in the alcohol detection apparatus 10A, the alcohol sensor 11 performs the initialization operation, for example, after the lock state of each of all the doors becomes the locked state. This makes it possible to perform the initialization operation at a timing when the environment is stabilized after, for example, impact, wind pressure, or an atmospheric pressure change in the vehicle is caused by opening and closing of the door. This helps to increase the detection accuracy of the alcohol component.

### Modification Example 2

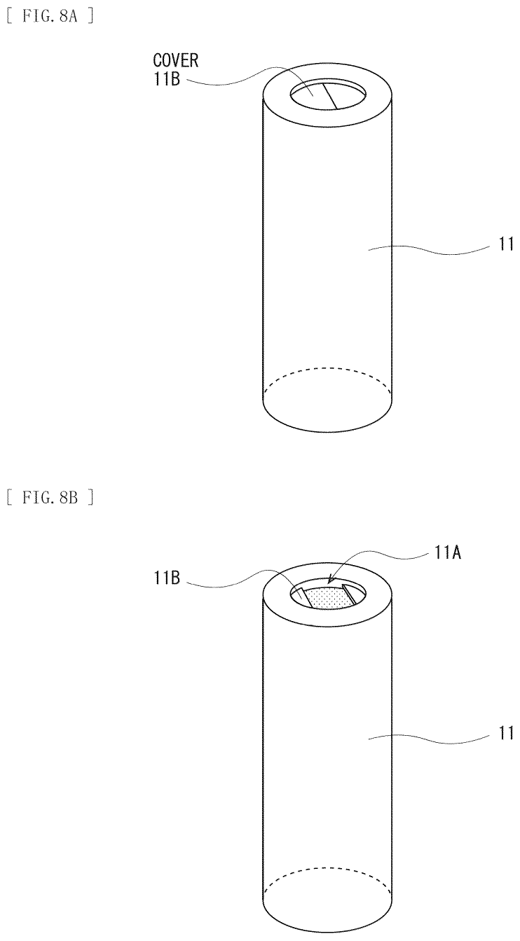

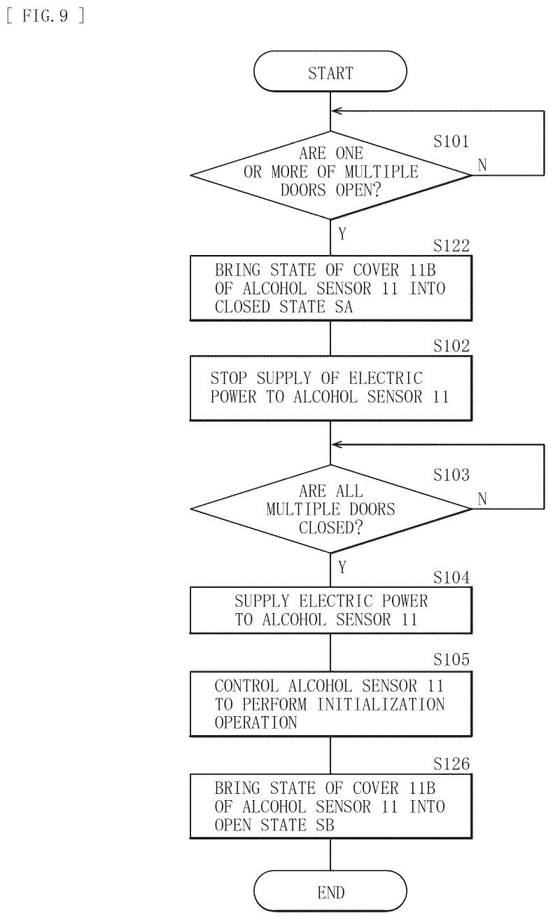

In the embodiment described above, the alcohol sensor 11 detects the alcohol component included in the exhaled breath of the driver blown to the inlet 11A. The alcohol sensor 11 may include a cover 11B configured to cover the inlet 11A as illustrated in FIGS. 8A and 8B. The cover 11B is configured to take either a state of being closed to cover the inlet 11A (a closed state SA) as illustrated in FIG. 8A or a state of being open to expose the inlet 11A (an open state SB) as illustrated in FIG. 8B. When the cover 11B is in the open state SB, the alcohol sensor 11 is able to detect, for example, the alcohol component included in the exhaled breath of the driver blown to the inlet 11A. When the cover 11B is in the closed state SA, the inlet 11A of the alcohol sensor 11 is not exposed, which makes it possible to reduce, for example, the possibility of rain entering inside the alcohol sensor 11. The state of the cover 11B is set based on, for example, an instruction from the alcohol sensor controller 12.

FIG. 9 illustrates an operation example of the alcohol detection apparatus 10 according to the modification example.

First, the alcohol sensor controller 12 according to the modification example determines whether one or more of the doors are open, based on the information regarding the open or closed state of each of the doors supplied from the vehicle controller 20 (step S101). If all the doors are closed (“N” in step S101), the alcohol sensor controller 12 repeats the process of step S101 until one or more of the doors are opened.

If one or more of the doors are open in step S101 (“Y” in step S101), the alcohol sensor controller 12 according to the modification example brings the state of the cover 11B of the alcohol sensor 11 into the closed state SA (step S122).

Next, the alcohol sensor controller 12 stops supply of electric power to the alcohol sensor 11 (step S102). In this manner, the operation mode of the alcohol detection apparatus 10A becomes the standby mode of waiting for detection of the alcohol component. In the standby mode, the alcohol sensor 11 is protectable to prevent, for example, rain from entering inside the alcohol sensor 11.

Next, the alcohol sensor controller 12 checks whether all the doors are closed, based on the information regarding the open or closed state of each of the doors supplied from the vehicle controller 20 (step S103). If one or more of the doors are still open (“N” in step S103), the alcohol sensor controller 12 repeats the process of step S103 until all the doors are closed.

If all the doors are closed in step S103 (“Y” in step S103), the alcohol sensor controller 12 supplies electric power to the alcohol sensor 11 (step S104).

Next, the alcohol sensor controller 12 controls the operation of the alcohol sensor 11 to cause the alcohol sensor 11 to perform the initialization operation (step S105). The alcohol sensor 11 is calibrated by the initialization operation to become able to detect the alcohol component at a high detection accuracy.

Thereafter, the alcohol sensor controller 12 brings the state of the cover 11B of the alcohol sensor 11 into the open state SB (step S126). In this manner, the operation mode of the alcohol detection apparatus 10 becomes the detection mode in which the alcohol component is detectable.

This is the end of the process.

Here, the inlet 11A corresponds to a specific example of an “inlet” in one embodiment of the disclosure. The cover 11B corresponds to a specific example of a “cover” in one embodiment of the disclosure. The closed state SA corresponds to a specific example of a “first state” in one embodiment of the disclosure. The open state SB corresponds to a specific example of a “second state” in one embodiment of the disclosure.

In the alcohol detection apparatus 10 according to the modification example, the alcohol sensor 11 includes the inlet 11A and the cover 11B. The inlet 11A is configured to allow the occupant to blow the exhaled breath into the inlet 11A. The cover 11B is provided at the inlet 11A and configured to take the closed state SA of covering the inlet 11A or the open state SB of exposing the inlet 11A. The alcohol sensor controller 12 is configured to bring the state of the cover into the closed state SA in the period in which one or more of the doors are in the open state, and bring the state of the cover into the open state SB in the period in which all the doors are in the closed state. Thus, for example, it is possible to reduce the possibility of rain entering inside the alcohol sensor 11, making it possible to protect the alcohol sensor 11.

### Modification Example 3

In the embodiment described above, the alcohol sensor 11 is mounted on the inner panel of the door 31, but the disclosure is not limited thereto. Alternatively, for example, the alcohol sensor 11 may be mounted inside the door 31. Specifically, for example, the alcohol sensor 11 may be mounted in a space between an outer panel opposed to the outside of the vehicle 1 and the inner panel opposed to the vehicle compartment of the vehicle 1. In another example, the alcohol sensor 11 may be detachable from the inner panel of the door 31. In another example, the alcohol sensor 11 may be mounted on another part inside the vehicle 1.

### Other Modification Examples

Two or more of these modification examples may be combined.

Although some example embodiments of the disclosure have been described in the foregoing by way of example with reference to the accompanying drawings, the disclosure is by no means limited to the embodiments described above. It should be appreciated that modifications and alterations may be made by persons skilled in the art without departing from the scope as defined by the appended claims. The disclosure is intended to include such modifications and alterations in so far as they fall within the scope of the appended claims or the equivalents thereof.

For example, in the above embodiment or the like, the technology is applied to the vehicle having a driver's seat on the right side, but the disclosure is not limited thereto. Alternatively, for example, the technology may be applied to a vehicle having a driver's seat on the left side.

The effects described herein are mere examples, and effects of the disclosure are therefore not limited to those described herein. Accordingly, the disclosure may achieve any other effect.

The disclosure may also encompass the following embodiments.
(1)

An alcohol detection apparatus including:
an alcohol sensor configured to detect an alcohol component included in exhaled breath of an occupant of a vehicle; and
a control circuit configured to, based on a detection result of a door sensor configured to detect an open or closed state of each of doors of the vehicle, control the alcohol sensor not to perform an initialization operation in a period in which one or more of the doors are in an open state, and control the alcohol sensor to perform the initialization operation after all the doors enter the closed state.
(2)

The alcohol detection apparatus according to (1), in which
the alcohol sensor includes
   an inlet configured to allow the occupant to blow the exhaled breath into the inlet, and
   a cover provided at the inlet and configured to take a first state of covering the inlet or a second state of exposing the inlet, and
the control circuit is configured to
   bring the state of the cover into the first state in the period in which one or more of the doors are in the open state, and
   bring the state of the cover into the second state in a period in which all the doors are in the closed state.
(3)

The alcohol detection apparatus according to (1) or (2), in which the control circuit is configured to
stop supply of electric power to the alcohol sensor in the period in which one or more of the doors are in the open state, and
supply the electric power to the alcohol sensor after all the doors enter the closed state.
(4)

An alcohol detection apparatus including:
an alcohol sensor configured to detect an alcohol component included in exhaled breath of an occupant of a vehicle; and
a control circuit configured to, based on a detection result of a door lock sensor configured to detect a lock state of each of doors of the vehicle, control the alcohol sensor not to perform an initialization operation in a period in which the lock state of each of one or more of the doors is an unlocked state, and control the alcohol sensor to perform the initialization operation after the lock state of each of all the doors becomes the locked state.
(5)

The alcohol detection apparatus according to (4), in which
the alcohol sensor includes
   an inlet configured to allow the occupant to blow the exhaled breath into the inlet, and
   a cover provided at the inlet and configured to take a first state of covering the inlet or a second state of exposing the inlet, and
the control circuit is configured to
   bring the state of the cover into the first state in the period in which the lock state of each of one or more of the doors is the unlocked state, and
   bring the state of the cover into the second state in a period in which the lock state of each of all the doors is the locked state.
(6)

The alcohol detection apparatus according to (4) or (5), in which the control circuit is configured to
stop supply of electric power to the alcohol sensor in the period in which the lock state of each of one or more of the doors is the unlocked state, and
supply the electric power to the alcohol sensor after the lock state of each of all the doors becomes the locked state.

The alcohol sensor controller 12 illustrated in FIG. 2 is implementable by circuitry including at least one semiconductor integrated circuit such as at least one processor (e.g., a central processing unit (CPU)), at least one application specific integrated circuit (ASIC), and/or at least one field programmable gate array (FPGA). At least one processor is configurable, by reading instructions from at least one machine readable non-transitory tangible medium, to perform all or a part of functions of the alcohol sensor controller 12 illustrated in FIG. 2. Such a medium may take many forms, including, but not limited to, any type of magnetic medium such as a hard disk, any type of optical medium such as a CD and a DVD, any type of semiconductor memory (i.e., semiconductor circuit) such as a volatile memory and a non-volatile memory. The volatile memory may include a DRAM and a SRAM, and the nonvolatile memory may include a ROM and a NVRAM. The ASIC is an integrated circuit (IC) customized to perform, and the FPGA is an integrated circuit designed to be configured after manufacturing in order to perform, all or a part of the functions of the alcohol sensor controller 12 illustrated in FIG. 2.

1. An alcohol detection apparatus comprising:
an alcohol sensor configured to detect an alcohol component included in exhaled breath of an occupant of a vehicle; and
a control circuit configured to, based on a detection result of a door sensor configured to detect an open or closed state of each of doors of the vehicle, control the alcohol sensor not to perform an initialization operation in a period in which one or more of the doors are in an open state, and control the alcohol sensor to perform the initialization operation after all the doors enter the closed state.

2. The alcohol detection apparatus according to claim 1, wherein
the alcohol sensor comprises
an inlet configured to allow the occupant to blow the exhaled breath into the inlet, and
a cover provided at the inlet and configured to take a first state of covering the inlet or a second state of exposing the inlet, and
the control circuit is configured to
bring a state of the cover into the first state in the period in which one or more of the doors are in the open state, and
bring the state of the cover into the second state in a period in which all the doors are in the closed state.

3. The alcohol detection apparatus according to claim 1, wherein the control circuit is configured to
stop supply of electric power to the alcohol sensor in the period in which one or more of the doors are in the open state, and
supply the electric power to the alcohol sensor after all the doors enter the closed state.

4. An alcohol detection apparatus comprising:
an alcohol sensor configured to detect an alcohol component included in exhaled breath of an occupant of a vehicle; and
a control circuit configured to, based on a detection result of a door lock sensor configured to detect a lock state of each of doors of the vehicle, control the alcohol sensor not to perform an initialization operation in a period in which the lock state of each of one or more of the doors is an unlocked state, and control the alcohol sensor to perform the initialization operation after the lock state of each of all the doors becomes a locked state.

5. The alcohol detection apparatus according to claim 4, wherein
the alcohol sensor comprises
an inlet configured to allow the occupant to blow the exhaled breath into the inlet, and
a cover provided at the inlet and configured to take a first state of covering the inlet or a second state of exposing the inlet, and
the control circuit is configured to
bring a state of the cover into the first state in the period in which the lock state of each of one or more of the doors is the unlocked state, and
bring the state of the cover into the second state in a period in which the lock state of each of all the doors is the locked state.

6. The alcohol detection apparatus according to claim 4, wherein the control circuit is configured to
stop supply of electric power to the alcohol sensor in the period in which the lock state of each of one or more of the doors is the unlocked state, and
supply the electric power to the alcohol sensor after the lock state of each of all the doors becomes the locked state.
